(19) United States
(12) Patent Application Publication
(10) Pub. No.: US 2024/0108852 A1
(43) Pub. Date: Apr. 4, 2024

(54) PERIPHERALLY INSERTED LEFT VENTRICULAR VENT AND ANTICOAGULATION CATHETER SYSTEM

(71) Applicant: Duke University

(72) Inventor: Carmelo A. Milano, Durham, NC (US)

(21) Appl. No.: 17/768,454

(22) PCT Filed: Oct. 14, 2020
(86) PCT No.: PCT/US2020/055586

§ 371 Date: Apr. 12, 2022

(51) Int. Cl. A61M 25/00; A61M 1/16; A61M 1/36

(52) U.S. Cl. CPC A61M 25/0026; A61M 1/1698; A61M 1/3659; A61M 25/0045

(57) ABSTRACT

A catheter system with two lumens (e.g., a major lumen and a minor lumen) that can be inserted peripherally into the left heart. The major lumen is used for venting, or blood volume removal, and the minor lumen is used to deliver a treatment substance directly to the left heart. In particular, the minor lumen can be used to deliver an anticoagulation medication directly to the left ventricle. The major lumen can be incorporated into an ECMO system.

### CROSS-REFERENCE TO RELATED APPLICATIONS

This application is a non-provisional of and claims benefit of U.S. Provisional Patent Application No. 62/914,631, filed on Oct. 14, 2019, the contents of which are incorporated herein by reference.

### BACKGROUND

Veno-arterial extra-corporeal membrane oxygenation (VA-ECMO) support is a rapidly growing technique to treat patients in cardiogenic shock. As its use increases, incidence of the common complications of support have become more widely characterized. Some of these complications result from abnormal blood flow through the left heart. During VA-ECMO support, the left ventricle (LV) has reduced blood flow, may have stagnant blood and can become distended. This in turn can lead to a number of associated adverse events. For example, increased LV pressure can be transmitted to the pulmonary circulation and cause pulmonary edema and other forms of injury to the lungs. The distended LV is less likely to experience functional recovery and stagnant blood can lead to thrombus formation, thromboembolism and stroke. One method of reducing LV distention is by “venting,” or the insertion of a catheter device directly into the left heart to remove the buildup of blood. Currently, however, such catheters may increase the risk of thrombus formation, as a foreign object may be a nidus for thrombus formation. Hence, there is an ongoing need for improved methods of LV venting and blood clot prevention.

Anticoagulation medications such as heparin or bivalirudin, which are used in conjunction with VA-ECMO, are administered intravenously. Delivery of anticoagulation medications directly into the left heart, can result in greater anticoagulant effects in the heart and will reduce thrombus formation

### SUMMARY

The present disclosure provides a catheter that can be inserted in the periphery, and vent the left ventricle to reduce distention to improve cardiac output with the added capability of directly delivering anti-coagulant medication, if required. The catheter provides anticoagulant delivery directly to the left ventricle and may improve outcomes by avoiding blood clots in the LV.

In some aspects, a catheter system for accessing a left ventricle of a heart is disclosed. The catheter system comprises a major lumen having a proximal opening and a distal opening and a minor lumen extending separately from the major lumen over a length of the major lumen, the minor lumen including a distal opening positioned at a distal region of the major lumen. The major lumen is configured to remove blood from the left ventricle, and the minor lumen is configured to enable measurement of left heart pressure and to deliver a treatment substance directly into the left ventricle.

In some embodiments, the major lumen is configured to be connected into an extra-corporeal membrane oxygenation support (ECMO) system. In other embodiments, the system comprises intake holes disposed in the distal region of the major lumen, and the minor lumen connection is offset from the intake holes. In some embodiments, a thrombo-resistant coating is disposed on at least a portion of a surface of the catheter (e.g., on an outer surface of the catheter).

In some aspects, a method of delivering an anticoagulation medication directly to a heart of a subject is disclosed. The method includes inserting a catheter system into the heart, the catheter system including a major lumen and a minor lumen, connecting the major lumen to a peripheral device for oxygenating blood from the heart, and delivering a treatment substance directly into the heart with the minor lumen.

In some embodiments, the method comprises venting the heart using the major lumen, and venting the heart is performed as an adjunct to VA-ECMO support. In some embodiments, the catheter system is inserted into the left ventricle by peripheral insertion.

In some embodiments, the treatment substance comprises an anticoagulation medication.

Other aspects of the invention will become apparent by consideration of the detailed description and accompanying drawings.

### BRIEF DESCRIPTION OF THE DRAWINGS

FIG. 1 is a schematic illustration of a heart showing various catheter insertion approaches and techniques.

FIG. 2 is a schematic illustration of a catheter system in accordance with one embodiment of the present disclosure.

FIG. 3 is a schematic illustration of the catheter system in accordance with one embodiment of the present disclosure.

FIGS. 4A and 4B illustrate cross-sectional views of a catheter system in accordance with an embodiment of the present disclosure.

FIG. 5 is a schematic illustration of the catheter system shown in FIGS. 2-3 connected to an ECMO system.

FIG. 6 is a schematic diagram illustrating the catheter system shown in FIGS. 2-3 including a housing with a flow meter.

FIG. 7 is a flow diagram illustrating a method of performing a LV ventilation procedure.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

For the purposes of promoting an understanding of the principles of the present disclosure, reference will now be made to various embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and non-human animals. The term “non-human animals” of the disclosure includes all vertebrates. e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient). In some embodiments, the subject comprises a human who is undergoing a left ventricle ventilation with a device as described herein.

Unless otherwise defined, all technical terms used herein have the same meaning as commonly understood by one of ordinary skill in the art to which this disclosure belongs.

Conventional left ventricle (LV) catheters are typically inserted into a subject's heart through a central thoracic incision. The present disclosure takes a different approach by allowing a peripheral insertion. While central insertion is still possible, the disclosed method can advantageously be provided through percutaneous delivery (e.g., subclavian or axillary arteries, or femoral arteries). This avoids the need for opening the chest cavity.

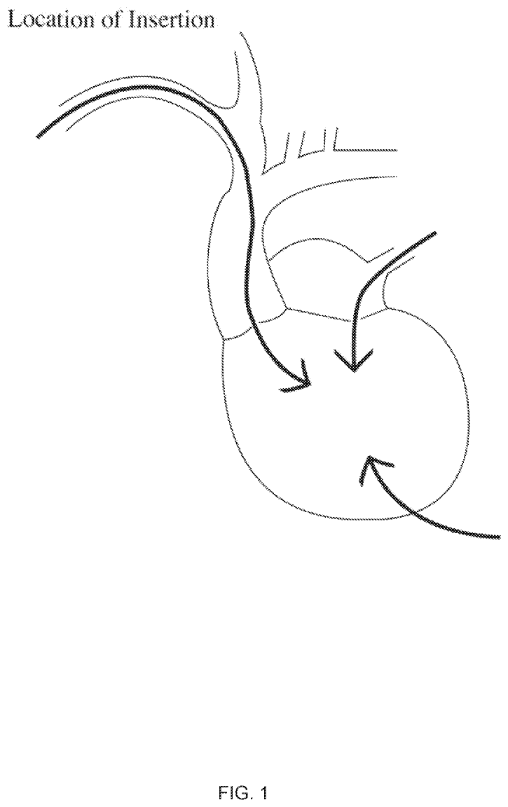

A peripherally inserted LV vent and anticoagulation system, referred to as a PILVAS catheter, is a catheter system which can be inserted via a peripheral artery without a thoracic incision. As illustrated in FIG. 1, the PILVAS catheter can be inserted either percutaneously (without an incision) or via an incision over and into a peripheral artery and then fluoroscopically positioned over a wire into the left heart through the aortic valve. The PILVAS system achieves at least three functions: left heart venting (i.e., removal of blood volume), left heart pressure transduction, and delivery of anticoagulation or other medication directly into the left heart.

A PILVAS catheter vents the left heart by evacuating blood from the left heart. In some embodiments, a PILVAS catheter can be used in conjunction with VA-ECMO. In these cases, the PILVAS catheter will vent blood from the left heart, which assists in preventing left heart distension, pressure overload, and subsequent back pressure into the pulmonary circulation. Preventing distension of the left heart, so called “unloading”, is widely believed to be beneficial to subsequent recovery of LV function.

A PILVAS catheter allows pressure transduction through a second lumen, described in further detail below. The second lumen can be connected to LV pressure measurement devices, which in turn can aid in the decision regarding ongoing venting of the left heart. For patients on VA-ECMO, a higher LV pressure could indicate need for venting.

In addition, the second lumen of the PILVAS catheter can enable delivery of coagulation-prevention substances directly into the left heart. For example, patients on VA-ECMO support have reduced blood flow through the left heart and are at risk for thrombus formation and subsequent stroke. Delivery of an anti-coagulant into the left heart can help prevent these adverse events.

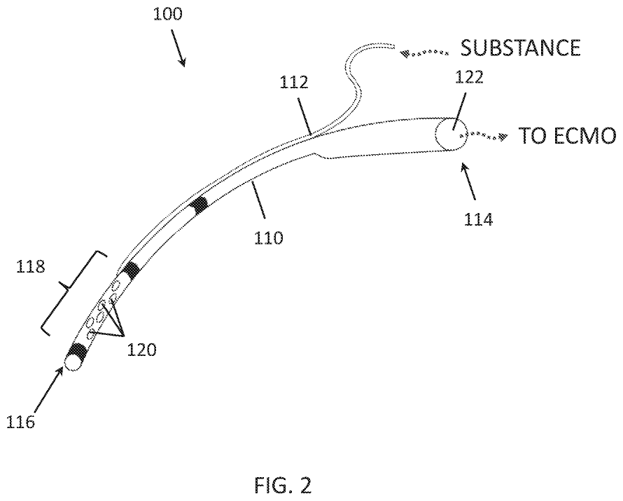

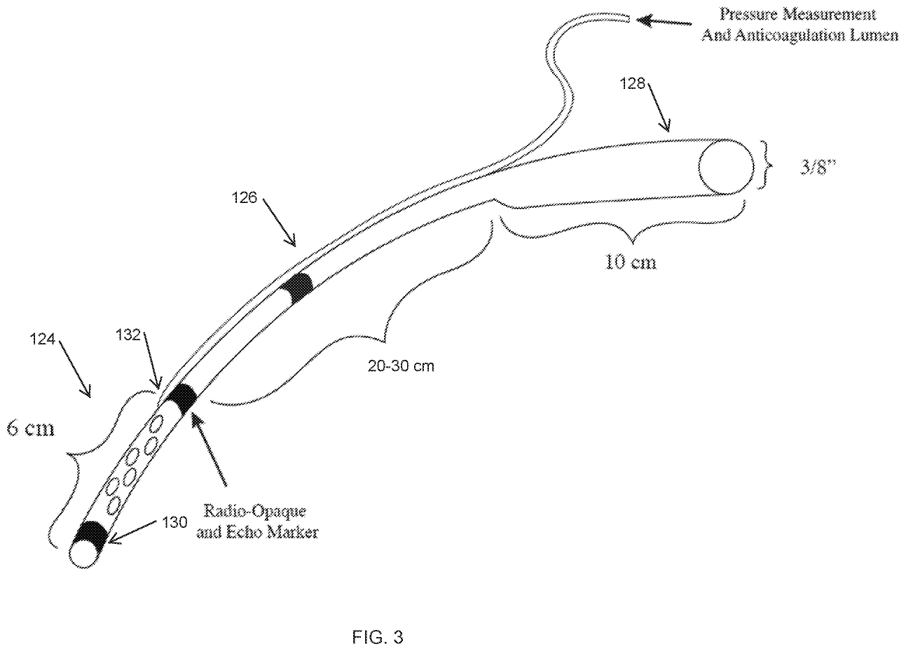

FIGS. 2-3 illustrate a PILVAS catheter 100 according to an embodiment of the present disclosure. The catheter 100 includes two lumens: a major lumen 110 and a minor lumen 112. The catheter 100 includes a proximal end 114 and a distal end 116. In one configuration, the major lumen 110 includes a first distal portion 124, a second intermediate portion 126, and a third proximal portion 128 or a step-up portion that is configured to connect to an ECMO circuit. The first portion 124 includes a first inner diameter, the second portion 126 includes a second inner diameter, and the third portion 128 includes a third inner diameter. The first inner diameter of the first portion 124 and the second inner diameter of the second portion 126 are less than the third inner diameter of the third portion 128. The first inner diameter and the second inner diameter may be the same, but also may vary in some embodiments. In general, the third inner diameter is about ⅜″ (e.g., 9.525 mm), and therefore the first inner diameter and the second inner diameter are less than ⅜″.

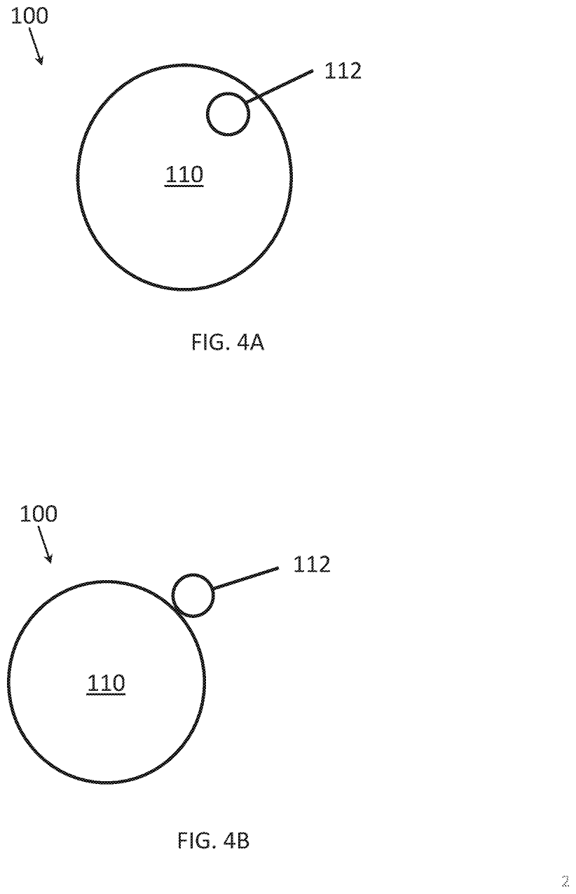

FIGS. 4A-4B illustrate a cross-sectional view of the catheter according to two constructions. As illustrated in FIG. 4A, a cross-section of a catheter constructed in accordance with the present disclosure shows the minor lumen 112 inside of the major lumen 110 whereas FIG. 4B illustrates a catheter with two separate hollow tubes that are coupled together as a single catheter. In the construction illustrated in FIG. 4A, the major lumen 110 includes a distal aperture and a proximal aperture where the minor lumen 112 exits the major lumen. For example, the distal end of the minor lumen 112 exits the distal aperture of the major lumen, and similarly, the proximal end of the minor lumen 112 exits the proximal aperture of the major lumen. This configuration allows the distal end of the minor lumen to deliver a substance to the left heart as further described below, and also allows the proximal end to separately connect to a peripheral device as described below.

In one example, a catheter 100 for axillary insertion is illustrated in FIG. 3. In this example, the first portion 124 of the major lumen 110 may comprise a length of about 6 cm, the second portion 126 of the major lumen 110 may comprise a length of about 20-30 cm, and the third portion 128 of the major lumen 110 may comprise a length of about 10 cm. The dimensions illustrated in FIG. 3 illustrate only one example and it is understood that the portions of the major lumen 110 may vary from the lengths and ratios shown. The lengths of the portions of the major lumen 110 may vary depending on the overall length of the catheter 100. Additionally, the overall length of the catheter 100 can vary depending on the insertion site. For example, a catheter for central/surgical insertion can be shorter (e.g., 30-35 cm), while a catheter for peripheral axillary and femoral insertion may be longer (e.g., 36-70 cm). Providing a variety of catheter lengths enables both central insertion into the heart and peripheral insertion.

It will be understood by a person of ordinary skill in the art that the exact size (e.g., a diameter) of the catheter 100 can vary depending on venting volume requirements, site of insertion, and patient size. The major lumen 110 is configured to withdraw or vent blood from the left heart and has an inner and an outer diameter greater than an inner diameter and an outer diameter of the minor lumen 112. In a non-limiting example embodiment, the outer diameter of the catheter 100 can be in the range of approximately 10-20 French (approximately 3-7 mm). In one particular embodiment, the outer diameter (or greatest extent) of the catheter 100 can be 14 French (approximately 4.67 mm). The inner diameter of the major lumen 110 can be approximately 2.5 mm to 4.0 mm. In some constructions, the inner diameter of the minor lumen 112 may be approximately 0.2 mm to 1.3 mm. In general, and in some constructions, the minor lumen 112 is about 5-10% of the cross-sectional area of the major lumen 110.

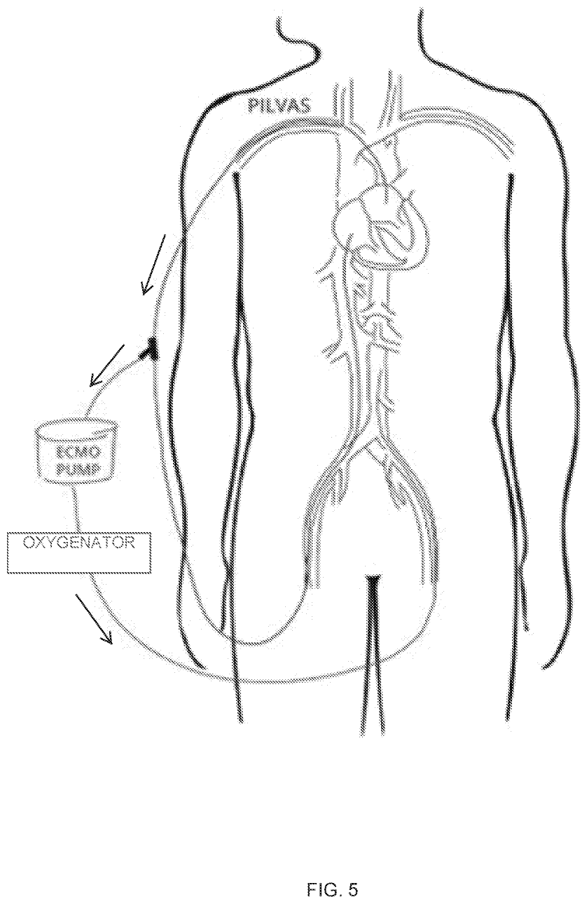

The major lumen 110 includes an outlet 122, which can be configured to interface with an ECMO circuit, as illustrated in FIG. 5, which typically uses ⅜″ tubing. The third proximal portion 128 of the major lumen 110 is configured to connect to the ECMO circuit for fluid communication therebetween. Typical volumes of which would be vented can, for example, range from approximately 200 ml to 2 liters per minute. In one example, the outer diameter of the major lumen 110 is 14 French to provide an evacuation of LV from 200 to 600 ml/min. In this example, a 14 Fr major lumen 110 was able to achieve adequate volume drainage in a sophisticated bench top mock loop of the human circulation on VA ECMO support. This is supportive evidence that the 14 Fr sized major lumen 110 can evacuate adequate LV volumes from 200 to 600 ml/min at VA ECMO simulated flows of 4.0 lpm.

With continued reference to FIG. 3, the major lumen 110 can include one or more bands 130 positioned on the outer surface of the major lumen 110. The band(s) 130 can comprise radio-opaque material for visualization while positioning the major lumen 110 during insertion guided with fluoroscopy or other imaging procedure. The first portion 124 of the major lumen 110 at the distal end 116 of the catheter 100 includes an intake area 118. The intake area 118 can optionally include several apertures 120, which would be positioned inside the left heart once the catheter 110 is inserted. The apertures 120 may be arranged circumferentially around the outer surface of the first portion 124. Alternatively, the apertures 120 may be arranged around a portion of the circumference and not extend 360 degrees around the outer surface of the first portion 124. The apertures 120 may include a circular or ovular shape or a combination thereof. As illustrated in FIG. 3, the first portion 124 includes six apertures 120, however fewer or more than six apertures may be accommodated. The sizes of the apertures 120 may vary and may have a total area that is equivalent to the inner cross-sectional area of the major lumen 110.

The minor lumen 112 is independent from but coupled or connected to the major lumen 110 and its distal end terminates near the proximal end of the intake area 118. The distal end of the minor lumen 112 includes an aperture 132 or hole. The aperture 132 may be at the tip of the minor lumen or on a radial surface of a wall of the minor lumen. The distal opening 132 provides fluid communication with the left heart (e.g., the LV). For example, the minor lumen 112 terminates between the first portion 124 and the second portion 126 of the major lumen 110. The minor lumen 112 terminates at the major lumen 110 at a location radially offset from the intake holes 120, in order to prevent immediate reuptake/recirculation of a treatment substance being delivered via the minor lumen. The opposite end of the minor lumen 112, i.e., the proximal end, includes a standard connector (e.g., Luer-type connector) for connection to additional tubing, a pressure measurement device or a delivery device, such as a syringe or IV infusion pump, for delivery of the treatment substance. Delivery rates of the treatment substance can be adjusted according to the type of solution and the treatment method. For example, in some instances an infusion rate of the treatment substance can be approximately 10 ml per hour.

The major lumen 110 includes a first axis that extends therethrough. The minor lumen 112 also includes a second axis that extends therethrough. The first axis and the second axis are oriented parallel to one another through at least a portion of their lengths.

The outer surfaces of the lumens 110, 112 can optionally be joined together or in contact with one another along their entire length or only a portion of their lengths. Optionally, the outer surface or surfaces of the catheter 100 can be at least partially coated, or substantially fully coated, with a surface treatment that is used to prevent the catheter from being a nidus for thrombus formation (e.g., a thrombo-resistant coating such as phosphatidylcholine). In other embodiments, the catheter system can include a valve (not shown) to relieve or reduce the negative pressure that can result in a collapsed ventricle. The valve would be configured such that air cannot be introduced into the system. In some embodiments the valve would be introduced into the portion of the catheter that is located outside of the body.

The minor lumen 112 serves at least two functions. First, the minor lumen 110 can be used to inject or deliver a substance, e.g., a therapeutic, to the left heart. In a non-limiting example, the substance can be an anticoagulation treatment drug. Other suitable treatment substances include thrombolytics, such as tissue plasminogen activator (TPA) and inotropic/pressor agents. Second, by pausing delivery of the substance, the minor lumen 112 can be connected to external systems and enables pressure measurement in the left heart.

In some embodiments, the major lumen 110 and the minor lumen 112 are non-compressible (e.g., formed from rigid or semi-rigid plastic material) but remain flexible to bend through the anatomy as it traverses the vascular system. It is further noted that the catheter 100 can comprise additional lumens without departing from the scope of the disclosure. For example, the catheter 100 can comprise a plurality of minor lumens 112 for simultaneously delivering multiple types of treatment substances or for performing additional monitoring functions.

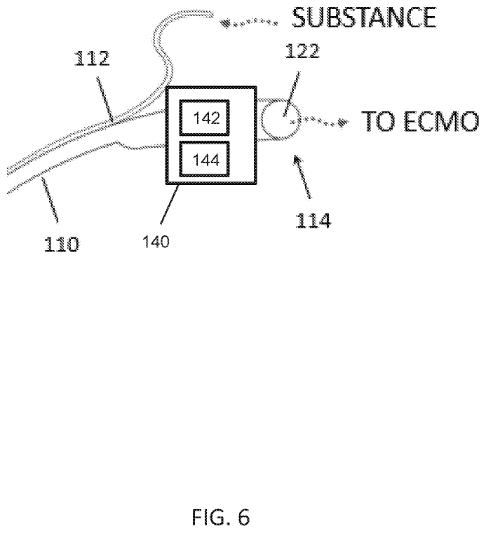

In another embodiment, as illustrated in FIG. 6, the catheter 100 may include a handle or housing 140 coupled to the major lumen 110. The housing 140 can include a flow meter 142 in fluid communication with the major lumen 110 and configured to measure volume of blood vented through the major lumen. The housing 140 can include a processor 144 for receiving data from the flow meter and a display for presenting the data in a user readable format.

The catheter 100 described above may be a component of a kit. The kit may include the catheter 100, a guidewire, an introducer, and other components suitable for peripheral arterial access and catheterization of the heart or other cardiac related surgical procedure. The introducer provides an interface between the guidewire and the catheter, enabling the catheter to be guided over the wire into final position. The guidewire and introducer are utilized for positioning and then are removed from the catheter enabling it to function as described herein. The guidewire can be radiopaque for image-guided positioning. The catheter 100 then travels along the guidewire to its position to provide therapy to and venting/evacuation of the LV.

Another embodiment of the present disclosure provides a method of performing a LV venting (e.g., evacuation of accumulated blood volume). With the system and method disclosed herein, a treatment substance can be delivered directly to the region of interest in a heart. In particular, an anticoagulation medication can be administered to the LV. This portion of the heart is susceptible to thrombosis, especially when occupied by a therapeutic device such as a catheter. The left heart is also susceptible to thrombosis when normal blood flow through it is diverted as is the case during support with VA ECMO. Unlike conventional methods, the disclosed method delivers anticoagulation medication directly to the heart, rather than delivery to a distal location. With the disclosed method, it is possible to localize the medication and therefore reduce the overall medication dose, while increasing the concentration at the treatment site. Specifically, direct infusion into the left heart can result in higher levels of anticoagulation in the left heart relative to the peripheral circulation. This can in turn reduce the risks associated with VA-ECMO, such as LV (and possibly left atrium (LA)) thrombus formation and thrombo-embolism, including stroke.

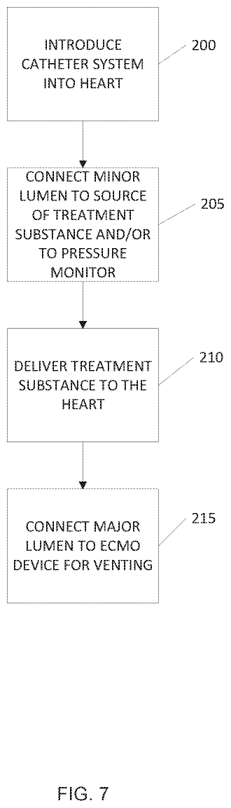

With reference to FIG. 7, in an initial step 200, a catheter system (e.g., the catheter 100), is introduced into the heart. This can be performed, for example, by a central insertion into the heart via a thoracic incision, or by a peripheral insertion via a peripheral vein or artery (typically femoral or axillary/brachial). Central insertion sites can include the left atrium via pulmonary vein cannulation and LV apical cannulation. In this case, the catheter can be surgically placed through purse string sutures in a manner similar to cardiac surgery cannulas, and the catheter can be fitted with a stylet to facilitate placement. Peripheral insertion is also possible. The catheter would be supplied as part of a kit including a needle, a guide wire, dilators, etc. The catheter can optionally be placed with assistance from fluoroscopic imaging. The catheter can be inserted into a vein and then guided into the left atrium via a septal puncture of the intra-atrial septum. Peripheral arterial insertion is also possible, for example by guiding the catheter system across the aortic valve into the LV.

In a subsequent step 205, the minor lumen 112 is connected to a source of a treatment substance. The treatment substance is delivered and administered at step 210 to the LV through the minor lumen (e.g., 112). This substance can be, for example, an anticoagulation treatment such as heparin or bivalirudin. Other possible medications that could be delivered into the left heart include thrombolytics, such as tissue plasminogen activator (TPA) and inotropic/pressor agents. However, it should be noted that, although the present disclosure is described in terms of an anticoagulation medication, any suitable treatment substance can be administered using the catheter system disclosed herein.

In a simultaneous or optional step 215, the major lumen 110 is connected to the ECMO device and operated to vent blood from the LV. In some embodiments, venting can be performed as an adjunct to VA-ECMO support.

Those skilled in the art will recognize improvements and modifications to the preferred implementations of the present disclosure. All such improvements and modifications are considered within the scope of the concepts disclosed herein and the claims that follow.

What is claimed is:

1. A catheter system for accessing a left ventricle of a heart, the catheter system comprising:
a major lumen having a proximal opening and a distal opening; and
a minor lumen extending separately from the major lumen over a length of the major lumen, the minor lumen including a distal opening positioned at a distal region of the major lumen,
wherein the major lumen is configured to remove blood from the left ventricle, and
wherein the minor lumen is configured to enable measurement of left heart pressure and to deliver a treatment substance directly into the left ventricle.

2. The system of claim 1, wherein the treatment substance comprises an anticoagulant.

3. The system of claim 1, wherein the major lumen is configured to be connected into an extra-corporeal membrane oxygenation support (ECMO) system.

4. The system of claim 1, comprising one or more intake apertures disposed in the distal region of the major lumen.

5. The system of claim 4, wherein the distal opening of the minor lumen is radially offset from the one or more intake apertures.

6. The system of claim 4, wherein the major lumen includes a first axis and wherein the one or more intake apertures includes a second axis oriented perpendicular to the first axis.

7. The system of claim 1, wherein the major lumen includes an outer surface, and further wherein the outer surface of the major lumen comprises a thrombo-resistant coating.

8. The system of claim 1, wherein the minor lumen includes an outer surface, and further wherein the outer surface of the minor lumen comprises a thrombo-resistant coating.

9. The system of claim 1, wherein the major lumen includes a first axis and the minor lumen includes a second axis, and wherein the first axis and the second axis are parallel along at least a portion of a length of the major lumen and the minor lumen.

10. The system of claim 1, wherein the major lumen includes a first diameter and the minor lumen includes a second diameter, and wherein the first diameter is greater than the second diameter.

11. The system of claim 1, wherein the major lumen includes a first portion having a first diameter and a second portion having a second diameter, and wherein the second diameter is greater than the first diameter.

12. The system of claim 1, wherein the major lumen includes a first axis and wherein the proximal opening is co-axial with the first axis.

13. A method of delivering a treatment substance directly to a heart of a subject, the method comprising:
inserting a catheter system into the heart, the catheter system including a major lumen and a minor lumen;
connecting the major lumen to a peripheral device for oxygenating blood from the heart; and
delivering a treatment substance directly into the heart with the minor lumen.

14. The method of claim 12, further comprising measuring pressure in the heart with the minor lumen.

15. The method of claim 12, comprising inserting the catheter system into the heart using a central insertion technique.

16. The method of claim 12, comprising inserting the catheter system into the heart using a peripheral insertion technique.

17. The method of claim 12, wherein the peripheral device is an ECMO system, and further comprising venting blood from the left heart using the major lumen.

18. The method of claim 12, wherein the treatment substance comprises an anticoagulant.
